(19) United States
(12) Patent Application Publication
(10) Pub. No.: US 2015/0333124 A1
(43) Pub. Date: Nov. 19, 2015

(54) EDGE HALOGENATION OF GRAPHENE MATERIALS

(71) Applicants: BASF SE, Ludwigshafen (DE); Max-Planck-Gesellschaft zur Förderung der Wissenschaften e.V., Muenchen (DE)

(72) Inventors: Tobias Hintermann, Therwil (CH); Klaus Muellen, Koln (DE); Xinliang Feng, Dresden (DE); Yuan-Zhi Tan, Mainz (DE)

(73) Assignees: BASF SE, Ludwigshafen (DE); MAX-PLANCK-GESELLSCHAFT ZUR FÖRDERUNG DER WISSENSCHAFTEN E.V., Muenchen (DE)

(21) Appl. No.: 14/652,514

(22) PCT Filed: Dec. 2, 2013
(86) PCT No.: PCT/IB2013/060563

§ 371 Date: Jun. 16, 2015

Related U.S. Application Data
(60) Provisional application No. 61/739,736, filed on Dec. 20, 2012.

(51) Int. Cl. H01L 29/16; H01B 1/04; C07C 17/02

(52) U.S. Cl. CPC H01L 29/1606; C07C 17/02; H01B 1/04

(57) ABSTRACT

The present invention relates to a process for edge-halogenation of a graphene material; wherein the graphene material, which is selected from graphene, a graphene nanoribbon, a graphene molecule, or a mixture thereof, is reacted with a halogen-donor compound in the presence of a Lewis acid, so as to obtain an edge-halogenated graphene material.

Graphene is a two-dimensional sheet of sp²-hybridized carbon, with long-range π-conjugation, which results in extraordinary thermal, mechanical, and electronic properties. For manipulating the physical and chemical properties of graphene materials, chemical functionalization is of great interest.

In principle, graphene materials can be chemically functionalized by two different approaches. According to a first approach, the aromatic basal plane is modified by addition reaction with C═C bonds. At present, this is the commonly favoured approach. Alternatively, chemical functionalization can be effected at the edge of the graphene material, thereby resulting in edge-functionalized graphene (e.g. substituting the edge-bonded residues by another chemical group). This approach is of particular relevance for those graphene materials which have confined dimensions either in just one direction of the plane (graphene nanoribbons) or in both directions of the plane (graphene molecules, i.e. very large polycyclic aromatic compounds).

Edge functionalization can significantly affect the properties of the final graphene material. For example, a graphene nanoribbon can be changed from p-type semiconducting behavior into n-type semiconducting behavior in a transistor device via substitution of the edge-bonded H-atoms by amino groups. Graphene materials which are edge-functionalized by halogen atoms would also be of great interest. With the presence of edge-bonded halogen atoms, optical and electronic properties of the graphene material can be modified.

However, well-defined and controllable edge functionalization of graphenes still remains a great challenge.

It is an object of the present invention to provide a process for chemical functionalization of graphene materials, wherein said chemical functionalization is effected with high yield but selectively takes place in specific areas of the graphene materials. It is also an object of the present invention to provide a graphene material with a high degree of chemical functionalization but still having a well-defined structure.

The object is solved by a process for edge-halogenation of a graphene material; wherein the graphene material, which is selected from a graphene, a graphene nanoribbon, a graphene molecule, or a mixture thereof, is reacted with a halogen-donor compound in the presence of a Lewis acid, so as to obtain an edge-halogenated graphene material.

In the present invention, it was realized that graphene materials such as graphene, graphene nanoribbons and graphene molecules can be halogenated very selectively at the edge (via at least partially substituting those residues R_{E} which are covalently bonded to the sp²-hybridized carbon atoms forming the edge of the starting graphene material), while suppressing very effectively any halogenation on the aromatic basal plane of the graphene material, and the degree of halogenation at the edge of the graphene material is very high and may even be quantitative (i.e. 100%).

In the present invention, the graphene materials to be subjected to the halogenation process (i.e. the starting graphene materials) are selected from graphene, graphene nanoribbons (GNR), and graphene molecules. As known to the skilled person, in all these graphene materials, sp²-hybridized carbon atoms form an extended single-layered aromatic basal plane and those sp²-hybridized carbon atoms which are located at the very periphery of the aromatic basal plane are forming the edge of the graphene material. So any of these graphene materials has an aromatic basal plane and an edge. To each of these sp²-hybridized carbon atoms forming the edge of the graphene material, a residue is covalently attached (i.e. edge-bonded residues R_{E}). However, graphene, graphene nanoribbons and graphene molecules differ in their in-plane dimensions. The aromatic basal plane of graphene may in practice extend in both directions from several nanometers up to several microns, whereas the aromatic basal plane of graphene nanoribbons is in the form of a strip typically having a width of less than 50 nm or even less than 10 nm. Typically, the aspect ratio of graphene nanoribbons (i.e. ratio of length to width) is at least 10. In the relevant technical field, the term “graphene molecule” is typically used for very large polycyclic aromatic compounds with dimensions of up to 10 nm, typically 5 nm or less. The term “graphene material” also encompasses those materials wherein some of the carbon atoms of the aromatic basal plane are replaced by heteroatoms.

If the graphene starting material is a graphene molecule, it can be a polycyclic aromatic compound having 8 to 200 fused aromatic rings, more preferably 13 to 91 fused aromatic rings; or 34 to 91 fused aromatic rings, or 50 to 91 fused aromatic rings.

Apart from aromatic rings located at the very periphery, any aromatic ring is fused to 2-6 aromatic neighbor rings. Typically, the graphene molecule comprises at least 3 aromatic rings, more preferably at least 5 or at least 7 aromatic rings, even more preferably at least 14 or at least 16 aromatic rings which are fused to 3-6 aromatic neighbor rings.

Preferably, the fused aromatic rings of the polycyclic aromatic compound are six-membered carbon rings. However, it is also possible, that at least some of the fused aromatic rings of the polycyclic aromatic compound are heterocyclic rings (e.g. nitrogen-containing heterocyclic rings or boron-containing heterocyclic rings), which can be five-membered or six-membered.

The edge-bonded residues R_{E} covalently attached to the edge of the graphene starting material (i.e. the graphene, the graphene nanoribbon, or the graphene molecule) are preferably selected from hydrogen, a substituted or unsubstituted alkyl group, a substituted or unsubstituted aryl group (e.g. a substituted or unsubstituted phenyl group), or any combination or mixture thereof. The alkyl group can be a C₁₋₁₂ alkyl group, more preferably a C₁₋₈ alkyl group. In a preferred embodiment, the alkyl group is a tertiary alkyl group such as a tert-butyl group or a tert-octyl group.

Preferably, the graphene molecule is selected from one or more of the following compounds (I) to (VII):

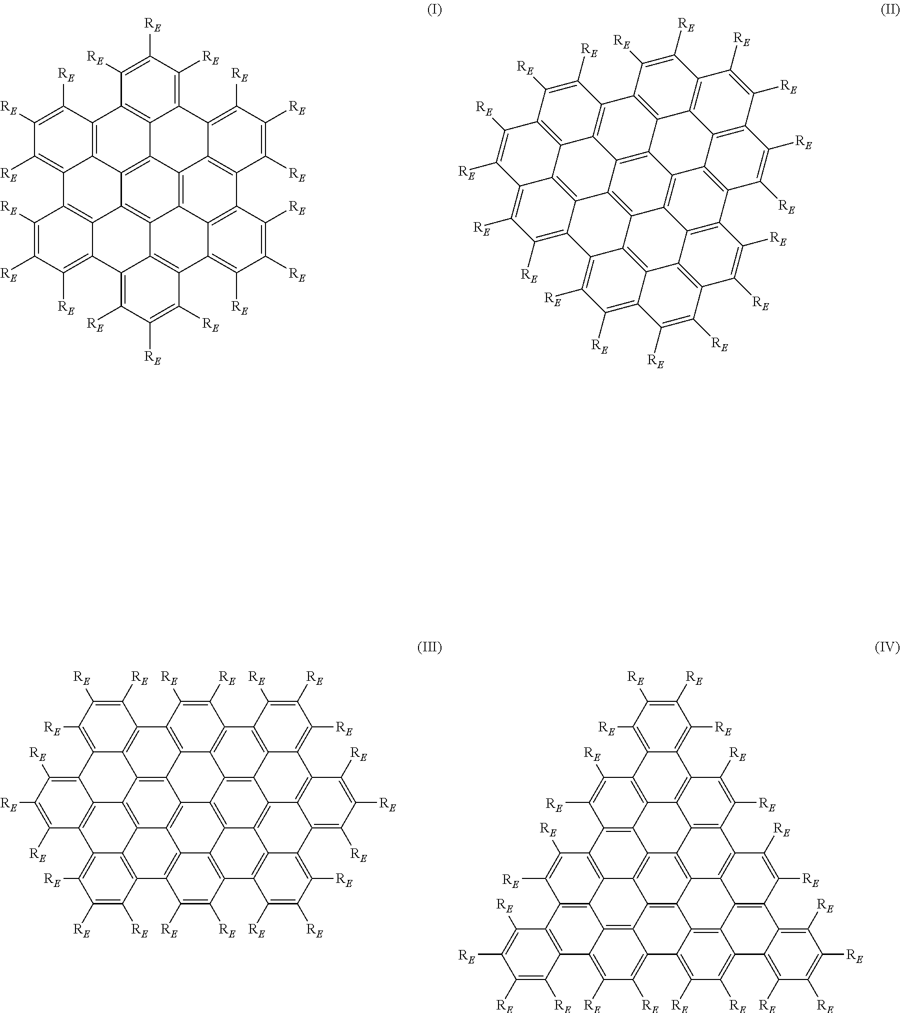

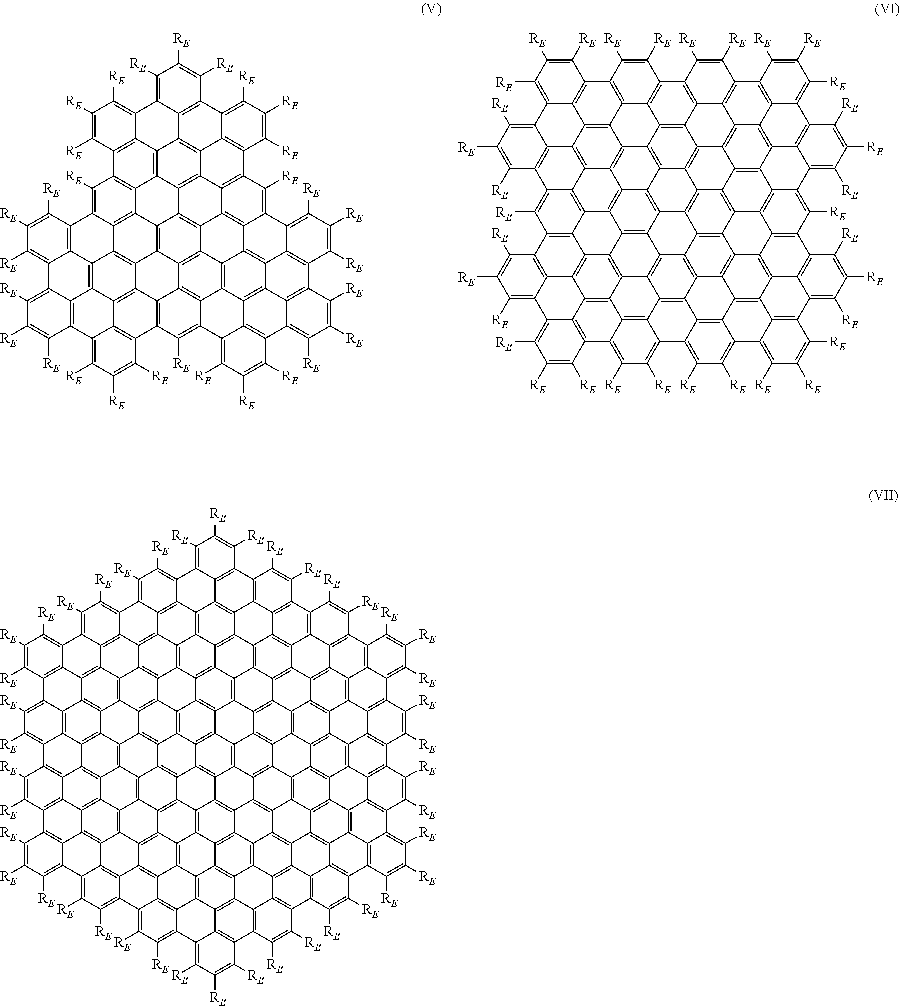

wherein the edge-bonded residues R_{E} have the same meaning as indicated above.

The graphene molecules to be subjected to the edge halogenation process of the present invention can be obtained by methods which are commonly known to the skilled person. The synthesis of such compounds is well described e.g. in the following literature. The preparation of compound I is described by K. Müllen et al. in J. Am. Chem. Soc. (2011) 133, 15221; or compound III in Angew. Chem. Int. Ed. (1998) 37, 2696; or compound IV in Angew. Chem. Int. Ed. (2007) 46, 3033; or compound VI in Angew. Chem. Int. Ed. (1997) 36, 631; or compound V and VII in Angew. Chem. Int. Ed. (1997) 36, 1604. Other syntheses by K. Müllen et al. are described e.g. in Carbon (1998) 36, 827; J. Am. Chem. Soc. (2000 122, 7707; J. Am. Chem. Soc. (2004) 126, 7794); J. Am. Chem. Soc. (2006), 128, 9526).

The graphene nanoribbons to be subjected to the edge halogenation process of the present invention can be obtained by methods which are commonly known to the skilled person. In general, the graphene nanoribbons can be prepared by top-down or bottom-up manufacturing methods.

Standard top-down fabrication techniques include cutting graphene sheets, e.g. by using lithography, unzipping of carbon nanotubes, as described in US2010/0047154 and US2011/0097258, or using nanowires as a template, as described in KR2011/005436.

Bottom-up approaches for preparing graphene nanoribbons are described e.g. by L. Dössel, L. Gherghel, X. Feng, K. Müllen, Angew. Chem. Int. Ed. 50, 2540-2543 (2011) and Cai, J.; et al. Nature 466, 470-473 (2010), as well as in PCT/EP2012/072445 and EP 12 169 326. By these bottom-up approaches, a graphene nanoribbon having a very well-defined structure is obtained. Similar to conventional polymers, a graphene nanoribbon prepared by such bottom-up approaches and therefore having a well-defined structure even on the “molecular” level, has its specific repeating unit. The term “repeating unit” relates to the part of the nanoribbon whose repetition would produce either the complete ribbon (except for the ends) or, if the GNR is made of two or more segments, one of these segments (except for the ends). The term “repeating unit” presupposes that there is at least one repetition of said unit.

Typically, the maximum width of the graphene nanoribbon is less than 50 nm, more preferably less than 10 nm.

The ratio of the maximum width of the graphene nanoribbon to its maximum length is preferably at least 10.

Width and length are measured with microscopic methods well known to those skilled in the art, such as atomic force microscopy (AFM), transmission electon microscopy, or scanning tunneling microscopy (STM). If resolution below a few nm is required (e.g. maximum width of GNR of less than 10 nm), STM is the method of choice and the apparent width is corrected for the finite tip radius by STM simulation as explained in J. Cai et al., Nature 466, pp. 470-473 (2010). The STM images are simulated according to the Tersoff-Hamann approach with an additional rolling ball algorithm to include tip effects on the apparent ribbon width. The integrated density of states between the Fermi energy and the Fermi energy plus a given sample bias are extracted from a Gaussian and plane waves approach for the given geometries.

The graphene to be subjected to the edge halogenation process of the present invention can be obtained by methods which are commonly known to the skilled person. A commonly used method is e.g. exfoliation of graphite by intercalation and/or applying mechanical forceAccording to another well-known preparation method, graphite is oxidized to graphite oxide which may then be exfoliated (e.g. by application of mechanical force, by ultrasonication, or in a basic medium) to graphene oxide, followed by reduction to graphene, e.g. by thermal treatment or by chemical reduction and/or applying a thermal shock treatment for exfoliation and reduction. (see e.g. W. Bielawski et al., Chem. Soc. Rev., 2010, 39, pp. 228-240).

As commonly known, the graphene, the graphene nanoribbons, or the graphene molecules can have a zig-zag edge structure, an armchair edge structure, or a combination of both. It is also known that the edge of graphene, graphene nanoribbons or graphene molecules may include the following structural element

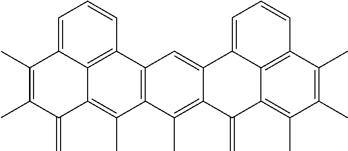

which is also referred to as a “double-fused bay edge configuration”. The graphene, the graphene nanoribbons, or the graphene molecules may include just one of these edge structures, or may have two or more edge sections which differ in edge structure.

Each of these edge structures outlined above (i.e. zigzag, armchair, and so-called “double-fused bay edge configuration”) can be subjected to the halogenation process of the present invention.

However, as will be discussed below in further detail, the “double-fused bay edge configuration” may include a “sterically protected” residue R_{E} which is not accessible to a halogen substitution, whereas the degree of halogenation in zig-zag and armchair edge structures in the process of the present invention is very high and can be close to or even equal to 100%.

According to the process of the present invention, the starting graphene material is reacted with a halogen-donor compound.

Halogen-donor compounds are generally known to the skilled person.

Preferably, the halogen-donor compound is selected from an interhalogen compound, S₂Cl₂, SOCl₂, a mixture of S₂Cl₂ and SOCl₂, SO₂Cl₂, Cl₂, Br₂, F₂, I₂, PCl₃, PCl₅, POCl₃, POCl₅, POBr₃, N-bromo succinimide, N-chloro succinimide, or any mixture thereof.

Preferably, the interhalogen compound is a compound having the following formula (VIII):

XYₙ (VIII)

wherein
n is 1, 3, 5, or 7;
X and Y, which are different, are selected from F, Cl, Br and I.

Preferably, X is of lower electronegativity than Y.

The interhalogen compound can be selected e.g. from ICl, IBr, BrF, BrCl, BrF₃, ClF, ClF₃, or any mixture thereof.

In a preferred embodiment, the halogen-donor compound is selected from ICl, S₂Cl₂, SOCl₂, a mixture of S₂Cl₂ and SOCl₂, Cl₂, or any mixture thereof.

Preferably, the halogenation process of the present invention is a chlorination process. Accordingly, it is preferred that the halogen-donor compound is a chlorine-donor (Cl-donor) compound.

If the halogen-donor compound is an interhalogen compound, it is typically the species of higher electronegativity which is substituting the edge-bonded residues R_{E} of the starting graphene material. To be more specific, if the starting graphene material is e.g. reacted with ICl, a chlorinated graphene material is obtained.

As indicated above, the starting graphene material and the halogen-donor compound are reacted in the presence of a Lewis acid.

In the present invention, the term “Lewis acid” is used according to its commonly accepted meaning and therefore relates to a molecular entity that is an electron-pair acceptor and therefore able to react with a Lewis base to form a Lewis adduct by sharing the electron pair furnished by the Lewis base.

The Lewis acid can be selected from a compound of formula (IX) or formula (X) or (XI) or (XII):

AX₃ (IX)
wherein A is Al, Fe, Sm, Sc, Hf, In, Y or B and X is halogen (preferably F, Cl, Br, P) or a trifluorosulfonate (e.g. trifluoromethanesulfonate OTf);

AX₅ (X)
wherein A is P, Sb, Mo or As and X is halogen (preferably Cl);

AX₄ (XI)
wherein A is Ti or Sn and X is halogen (preferably Cl);

AX₂ (XII)
wherein A is Mg, Zn, Cu or Be and X is halogen (preferably Cl) or a trifluorosulfonate (e.g. trifluoromethanesulfonate OTf).

Preferred Lewis acids include e.g. AlCl₃, AlBr₃, FeCl₃, FeBr₃, Sm(OTf)₃, BF₃, Cu(OTf)₂, ZnCl₂, BCl₃, BeCl₂, or any mixture thereof.

Preferably, the Lewis acid is acting as a catalyst. Accordingly, it is preferred to add the Lewis acid in low amounts.

The weight ratio of the graphene, the graphene nanoribbons or the graphene molecules to the Lewis acid can be varied over a broad range such as from 20/1 to 1/10, more preferably from 5/1 to 1/4.

The molar ratio of the edge-bonded residues R_{E} of the graphene, the graphene nanoribbons or the graphene molecules to the Lewis acid can be varied over a broad range such as from 100/1 to 1/5, more preferably from 25/1 to 1/2.

The weight ratio of the graphene, the graphene nanoribbons or the graphene molecules to the halogen-donor compound can be varied over a broad range such as from 1/1000 to 1/10, more preferably from 1/500 to 1/30.

The molar ratio of the edge-bonded residues R_{E} of the graphene, the graphene nanoribbons or the graphene molecules to the halogen-donor compound can be varied over a broad range such as from 1/1 to 1/200, more preferably from 1/5 to 1/70.

Preferably, the halogenation process of the present invention is carried out in an organic liquid or solvent.

Appropriate organic liquids or solvents are generally known to the skilled person and may include e.g. liquid hydrocarbons such as pentane, hexane, heptane, octane, or mixtures therof, or preferably halocarbons such as CCl₄, CHCl₃, CH₂Cl₂, dichloroethane, tetrachloroethane, CH₃Br, chlorobenzene, dichlorobenzene, chlorofluorocarbons, hydrochlorofluorocarbons, bromochlorofluorocarbons, bromofluorocarbons, hydrofluorocarbons, or any mixture thereof. The halogen donor compound can also be used as a liquid or solvent, e.g. SOCl₂ can be used as a liquid.

The reaction temperature can be varied over a broad range. An appropriate reaction temperature is e.g. in the range of from −20° C. to 200° C., more preferably 40° C. to 150° C. Depending on the type of liquid used, the upper limit of the reaction temperature may vary. The reaction temperature can be within the range of from −20° C. to the boiling point of the liquid or liquid mixture

In the halogenation process of the present invention, the graphene or graphene nanoribbons or graphene molecules and the halogen-donor compound and the Lewis acid can be added to the organic liquid in any order, preferably at room temperature, followed by sufficiently increasing the temperature so as to accelerate the edge halogenation reaction (i.e. substitution of the edge-bonded residues R_{E} by halogen atoms such as Cl). As already mentioned above, the reaction may be carried out under reflux or at least a temperature which is close to the boiling point T_{B} (under atmospheric pressure) of the liquid, e.g. T_{reaction} is 0.8*T_{B} to 1.0*T_{B}.

The reaction mixture is held at the reaction temperature for a time which is sufficient to provide a maximum degree of edge halogenation.

With the process of the present invention, it is possible to accomplish a high degree of edge halogenation, whereas any halogenation of the aromatic basal plane is more or less completely suppressed. Except for the double fused bay edge configuration which contains a sterically protected residue R_{E}, the degree of halogenation at the edge of the graphene materials is quantitative (i.e. 100% substitution of edge-bonded residues R_{E} by halogen atoms) or at least close to 100%, such as at least 90%, more preferably at least 94%, or at least 98%.

Only those edge-bonded residues R_{E} which are within sterically protected areas of specific edge configurations may not be accessible to a substitution by halogen atoms.

As already mentioned above, the graphene material subjected to the halogenation process of the present invention may have an edge or at least one edge section of the following structure (sometimes referred to as “double-fused bay edge”):

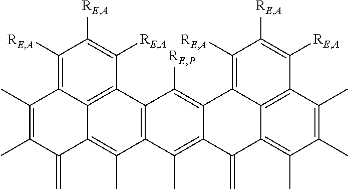

This double-fused bay edge structure has residues which are accessible to halogen substitution (in the above structure indicated as “R_{E,A}”), but also includes a “sterically protected” residue which is not accessible to halogen substitution (in the above structure indicated as “R_{E,P}”). In the halogenation process of the present invention, the degree of substitution of residues R_{E,A} by halogen is very high and can be almost quantitative or even equal to 100%. On the other hand, residues R_{E,P} are typically still present after completion of the halogenation process. So, even if the graphene starting material subjected to the halogenation process of the present invention includes a double-fused bay edge structure, an edge-halogenated graphene material having a well-defined substitution pattern is obtained, as there is more or less quantitative halogen substitution of residues R_{E,A} and no halogen substitution of residues R_{E,P}.

As will be discussed below in further detail and demonstrated by the Examples, the process of the present invention is selectively halogenating the edge of the starting graphene materials (via substitution of the edge-bonded residues R_{E} (i.e. the residues R_{E} which are bonded to the sp²-hybridized carbon atoms forming the edge of the graphene material) by halogen), whereas any halogenation of the aromatic basal plane is more or less completely suppressed. In other words, the hybridization state of the atoms which form the extended aromatic system of the graphene material does not change during the process of the present invention, as chemical functionalization is restricted to the edge. This still holds true even for extended reaction time and/or increased reaction temperature and/or excess of halogenating agent.

The degree of halogenation can be monitored by commonly known analytical methods, such as ¹H-NMR spectroscopy, ¹³C-NMR spectroscopy, XPS (X-ray photoelectron spectroscopy), IR spectroscopy and/or mass spectroscopy (e.g. matrix-assisted laser desorption/ionization time of flight (MALDI-TOF) mass spectroscopy).

If the desired degree of halogenation is achieved, the edge-halogenated graphene material can be separated from the reaction medium by commonly known methods such as filtration or evaporation of volatile components under reduced pressure. If needed, it is also possible to quench the halogenation reaction, e.g. by precipitation via addition of polar solvents such as ethanol.

The halogenated graphene materials obtained by the process of the present invention have improved solubility compared to graphenes. Just as an example, the halogenated graphene molecules prepared by the process of the present invention can be readily dissolved in common organic solvents such as toluene, chloroform and carbon disulfide so as to form a homogeneous solution.

Furthermore, as the halogenation process is selectively taking place at the edge but not on the aromatic basal plane, the electronic and optical properties of the graphene material can be modified and fine-tuned in a well-defined manner.

As discussed above, it is possible with the process of the present invention to provide a graphene material (i.e. a graphene, a graphene nanoribbon GNR, or a graphene molecule) which is selectively halogenated at the edge whereas halogenation on the aromatic basal plane of the graphene material is more or less completely suppressed.

So, according to a further aspect, the present invention provides a halogenated graphene material comprising an aromatic basal plane and an edge, wherein at least 65 mole % of the residues R_{E} covalently attached to the edge of the graphene material are halogen atoms HA_{E}, and the edge-bonded halogen atoms HA_{E} represent at least 95 mole % of all halogen atoms being present in the halogenated graphene material, and wherein the graphene material is selected from graphene, graphene nanoribbons and graphene molecules.

The ratio of edge-bonded halogen atoms to basal plane bonded halogen atoms, and the degree of halogen substitution at the edge of the graphene materials can be determined by known analytical methods. According to a preferred embodiment, XPS (X-ray photoelectron spectroscopy) analysis is used. In the present invention, XPS spectra were measured on an ESCALAB 250 (Thermo-VG Scientific) equipped with an Al Kα monochromatic source using powder sample.

With regard to the properties of the graphene molecule, the graphene nanoribbons and the graphene, reference can be made to the statements made above, of course with the exception of the edge-bonded residues R_{E} which are now mainly halogen atoms.

As explained above and known to the skilled person, in graphene materials, sp²-hybridized carbon atoms form an extended single-layered aromatic basal plane and those sp²-hybridized carbon atoms which are located at the very periphery of the aromatic basal plane are forming the edge of the graphene material. So, any of these graphene materials has an aromatic basal plane and an edge. To each of these sp²-hybridized carbon atoms forming the edge of the graphene material, a residue is covalently attached (i.e. edge-bonded residues R_{E}). With the present invention, it is possible to provide a graphene material wherein at least 65 mole % of the residues R_{E} covalently attached to the edge of the graphene material are halogen atoms HA_{E}, and the edge-bonded halogen atoms HA_{E} represent at least 95 mole % of all halogen atoms being present in the halogenated graphene material.

The graphene molecule can be a polycyclic aromatic compound having 8 to 200 fused aromatic rings, more preferably 13 to 91 fused aromatic rings; or 34 to 91 fused aromatic rings, or 50 to 91 fused aromatic rings. Apart from aromatic rings located at the very periphery, any aromatic ring is fused to 2-6 aromatic neighbor rings. Typically, the graphene molecule comprises at least 3 aromatic rings, more preferably at least 5 or at least 7 aromatic rings, even more preferably at least 14 or at least 16 aromatic rings which are fused to 3-6 aromatic neighbor rings. Preferably, the fused aromatic rings of the polycyclic aromatic compound are six-membered carbon rings. However, it is also possible, that at least some of the fused aromatic rings of the polycyclic aromatic compound are heterocyclic rings (e.g. nitrogen-containing or boron-containing heterocyclic rings), which can be five-membered or six-membered.

In a preferred embodiment, the halogenated graphene molecule has one of the following formulas (XIII), (XIV), (XV), (XVI), (XVII), (XVIII), and (XIX):

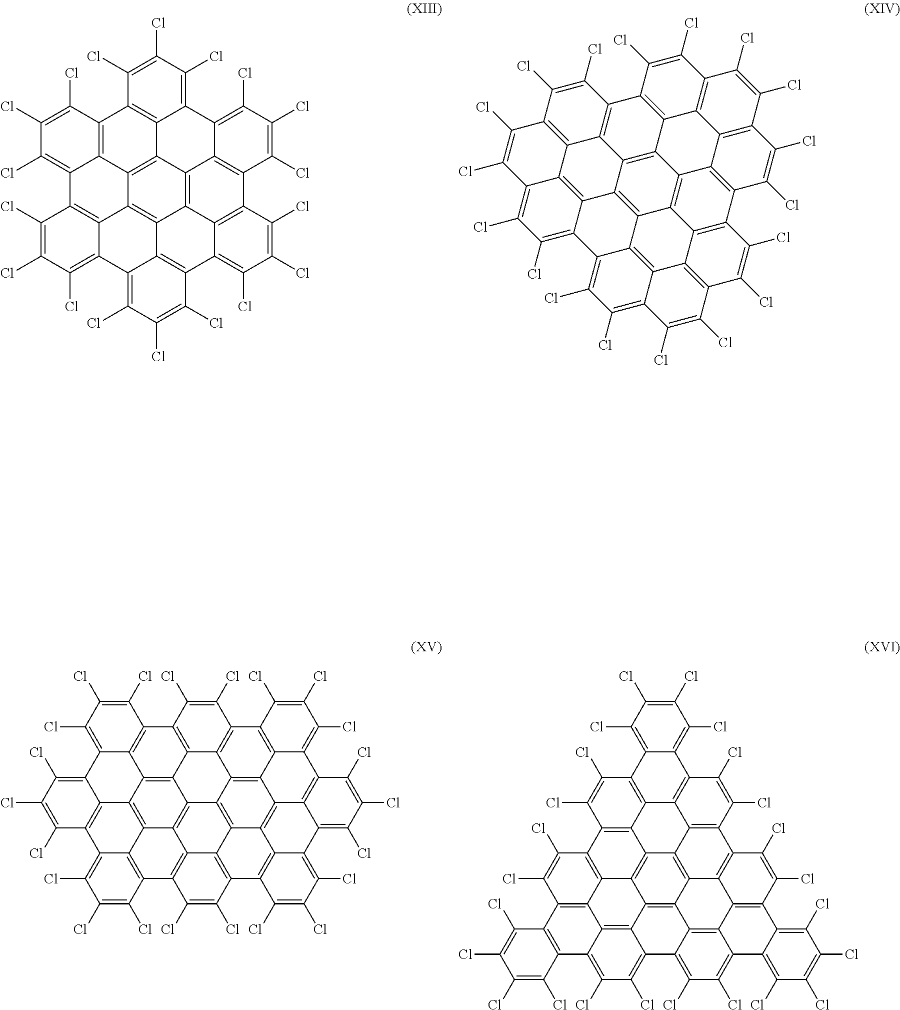

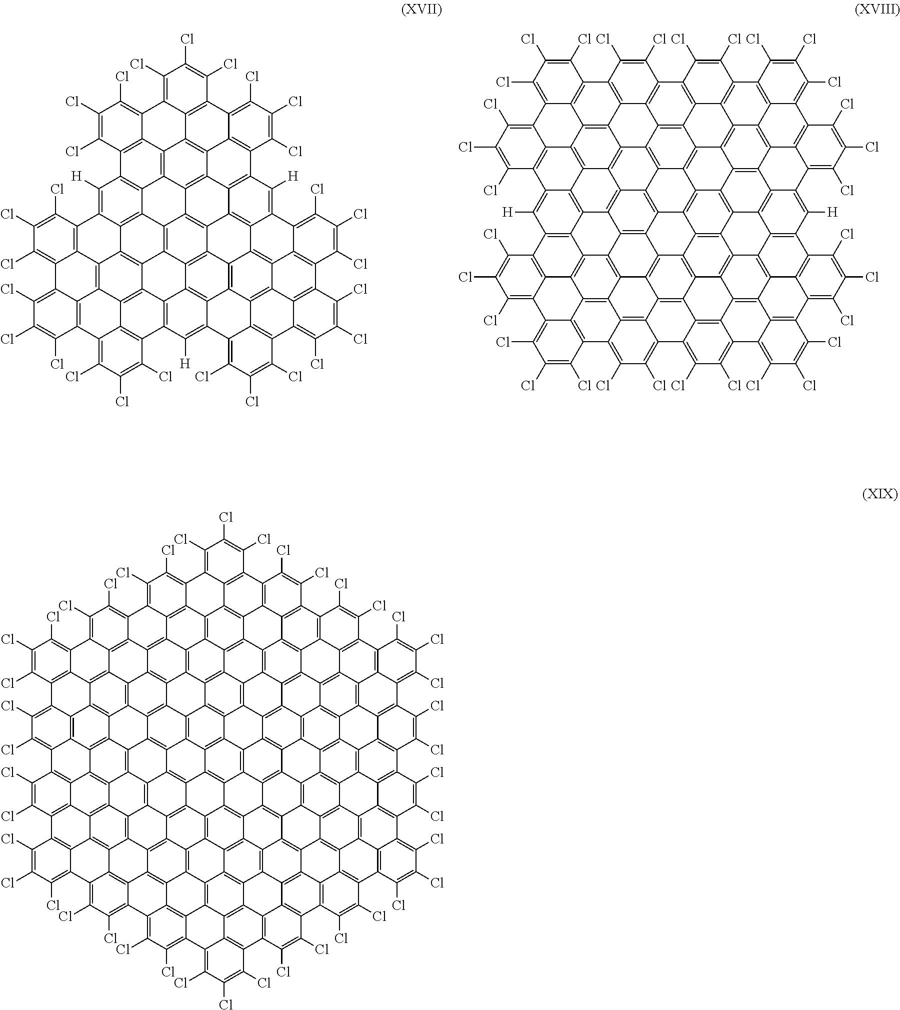

The chemical formula of the halogenated graphene molecule (XIII) is C₄₂Cl₁₈.

The chemical formula of the halogenated graphene molecule (XIV) is C₄₈Cl₁₈.

The chemical formula of the halogenated graphene molecule (XV) is C₆₀Cl₂₂.

The chemical formula of the halogenated graphene molecule (XVI) is C₆₀Cl₂₄.

The chemical formula of the halogenated graphene molecule (XVII) is C₉₆Cl₂₇H₃.

The chemical formula of the halogenated graphene molecule (XVIII) is C₁₃₂Cl₃₂H₂.

The chemical formula of the halogenated graphene molecule (XIX) is C₂₂₂CL₁₂.

Due to the high degree of halogenation, the graphene molecules of the present invention can be readily dissolved in common organic solvents such as toluene, chloroform and carbon disulfide. By commonly known methods such as solvent evaporation, the graphene molecules can be provided in a crystalline form.

If the halogenated graphene material is a halogenated graphene nanoribbon, its maximum width is typically less than 50 nm, more preferably less than 10 nm. The ratio of the maximum width of the graphene nanoribbon to its maximum length is preferably at least 10. Width and length are measured with microscopic methods such as atomic force microscopy (AFM), transmission electon microscopy, or scanning tunneling microscopy (STM). If resolution below a few nm is required (e.g. GNR with maximum width of less than 10 nm), STM is the method of choice and the apparent width is corrected for the finite tip radius by STM simulation as explained in J. Cai et al., Nature 466, pp. 470-473 (2010). The STM images are simulated according to the Tersoff-Hamann approach with an additional rolling ball algorithm to include tip effects on the apparent ribbon width. The integrated density of states between the Fermi energy and the Fermi energy plus a given sample bias are extracted from a Gaussian and plane waves approach for the given geometries.

As mentioned above, the graphene nanoribbon subjected to the halogenation process of the present invention may have a very well-defined structure even on the “molecular level” and therefore, similar to conventional polymers, be characterized by a specific repeating unit. Accordingly, as the process of the present invention results in a defined edge-halogenation, a halogenated graphene nanoribbon is obtained which comprises a repeating unit RU

Thus, in a preferred embodiment, the halogenated graphene material is a halogenated graphene nanoribbon which comprises a repeating unit RU, and the halogenated graphene nanoribbon or at least a segment thereof is made of [RU]ₙ, wherein 2≦n≦2500, more preferably 10≦n≦2500.

As indicated above, at least 65 mole % of the residues R_{E} covalently attached to the edge of the graphene material are halogen atoms HA_{E}, and the edge-bonded halogen atoms HA_{E} represent at least 95 mole % of all halogen atoms being present in the halogenated graphene material.

If the graphene material does not include any edge sections of the following structure (“double-fused bay edge configuration”):

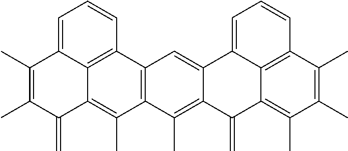

or includes such double-fused bay edge sections in a low amount, it is possible that the edge-bonded residues R_{E} are predominantly halogen atoms. Thus, in a preferred embodiment, at least 90 mole %, more preferably at least 95 mole %, even more preferably at least 98 mole % or even 100 mole % of the residues R_{E} covalently attached to the edge of the graphene material are halogen atoms HA_{E}.

On the other hand, if the edge of the graphene material is made of such a double-fused bay edge configuration only or includes said edge configuration in a high amount, the minimum amount of halogen atoms within the edge-bonded residues R_{E} is somewhat lower but is still at least 65 mole %, more preferably at least 70 mole % or at least 75 mole %.

Preferably, the edge-bonded halogen atoms HA_{E} represent at least 95 mole % or 98 mole %, more preferably at least 99 mole %, even more preferably 100 mole % of all halogen atoms being present in the halogenated graphene material.

According to a further aspect, the present invention provides a halogenated graphene material which is obtainable by the process for edge-halogenation of a graphene material as described above. Preferably, the halogenated graphene material obtainable by said process has the properties as described above. Reference can be made to the halogenated graphene molecules (in particular those of formulas (XIII) to (XIX)), the halogenated graphene nanoribbons (e.g. those of defined structure which are characterized by a repeating unit), and the halogenated graphene described above.

As mentioned above, due to the high degree of selective edge-halogenation, a halogenated graphene material is obtained which shows improved solubility or dispersibility in a liquid medium, in particular in an organic liquid medium such as toluene, chloroform, and carbon disulfide. The graphene material thus obtained can therefore easily be subjected to further transformations, e.g chemical modifications within the graphene basal plane or partial or complete substitution of the halogen at the edges.

Thus, according to a further aspect, the present invention provides a composition comprising one or more halogenated graphene materials as described above, which are dissolved or dispersed in a liquid medium, in particular an organic liquid medium.

Furthermore, as the halogenation process is selectively taking place at the edge but not on the aromatic basal plane, the electronic and optical properties of the graphene material can be modified and fine-tuned in a well-defined manner.

Thus, according to a further aspect, the present invention provides an electronic, optical, or optoelectronic device which comprises a semiconductor film (e.g. a thin film) comprising one or more of the halogenated graphene materials as described above.

Preferably, the device is an organic field effect transistor device, an organic photovoltaic device, or an organic light-emitting diode.

According to a further aspect, the present invention relates to the use of the halogenated graphene materials described above in an electronic, optical, or optoelectronic device, such as an organic field effect transistor device, an organic photovoltaic device, or an organic light-emitting diode.

The invention will now be described in further detail by the following Examples.

### EXAMPLES

### I. Preparation of Halogenated Graphene Molecules

The following graphene molecules were used as graphene starting materials:
C₄₂H₁₈ (graphene molecule of formula I)

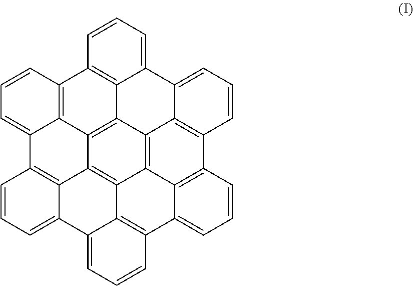 [SMILES: c1cc2c3cccc4c5cccc6c7cccc8c9cccc%10c%11cccc%12c(c1)c2c1c(c34)c(c56)c(c78)c(c%109)c1c%12%11]

The compound of formula C₄₂H₁₈ was prepared as described in K. Müllen et al. in J. Am. Chem. Soc. (2011) 133, 15221.
C₄₈H₁₈ (graphene molecule of formula II)

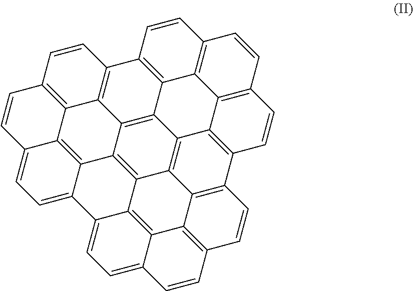 [SMILES: c1cc2ccc3c4ccc5ccc6ccc7c8ccc9ccc%10ccc%11c%12ccc1c1c2c3c2c(c%121)c1c%11c%10c9c8c1c1c7c6c5c4c21]

The compound of formula C₄₈H₁₈ was prepared K. Müllen et al. in J. Am. Chem. Soc. (2006) 128, 9526.
C₆₀H₂₂ (graphene molecule of formula III)

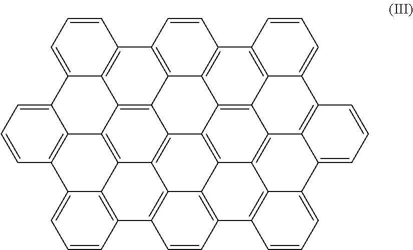 [SMILES: c1cc2c3cccc4c5ccc6c7cccc8c9cccc%10c%11cccc%12c%13ccc%14c%15cccc%16c(c1)c2c1c(c34)c2c5c6c3c(c87)c(c9%10)c(c%11%12)c4c%13c%14c(c1c%16%15)c2c34]

The compound of formula C₆₀H₂₂ was prepared as described in Angew. Chem. Int. Ed. (1998) 37, 2696.
C₆₀H₂₄ (graphene molecule of formula IV)

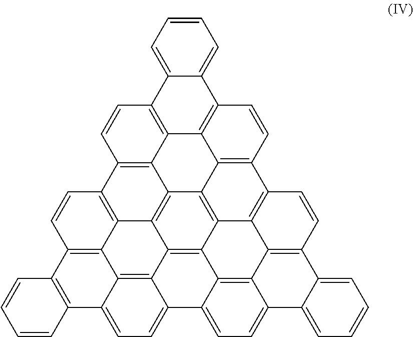 [SMILES: c1ccc2c(c1)c1ccc3c4ccc5c6ccccc6c6ccc7c8ccc9c%10ccccc%10c%10ccc%11c%12ccc2c2c1c3c1c(c%122)c2c%11c%10c9c8c2c2c7c6c5c4c12]

The compound of formula C₆₀H₂₄ was prepared as described in Angew. Chem. Int. Ed. (2007) 46, 3033.
C₉₆H₃₀ (graphene molecule of formula V)

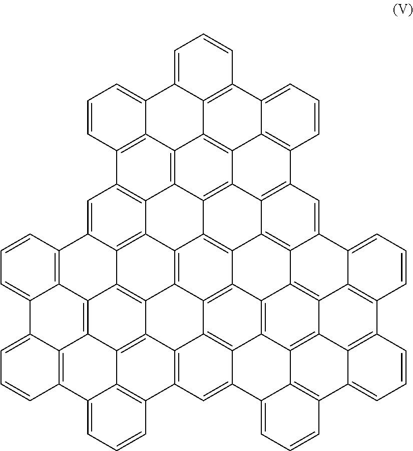 [SMILES: c1cc2c3cccc4c5cc6c7cccc8c9cccc%10c%11cccc%12c%13cc%14c%15cccc%16c%17cccc%18c%19cccc%20c%21cc%22c%23cccc%24c(c1)c2c1c(c34)c2c5c3c6c4c(c87)c(c9%10)c(c%11%12)c5c%13c6c%14c7c(c%16%15)c(c%18%17)c(c%19%20)c8c%21c9c%22c(c1c%24%23)c2c1c3c(c45)c6c(c87)c91]

The compound of formula C₉₆H₃₀ was prepared as described in Angew. Chem. Int. Ed. (1997) 36, 1604.
C₁₃₂H₃₄ (graphene molecule of formula VI)

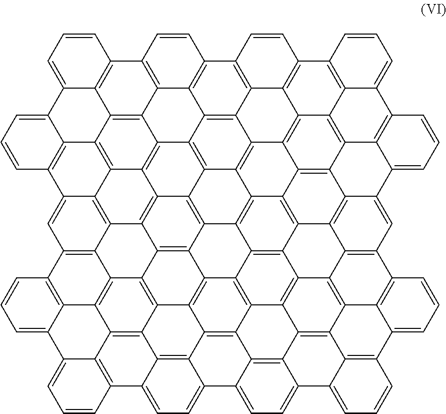 [SMILES: c1cc2c3cccc4c5cc6c7cccc8c9cccc%10c%11ccc%12c%13ccc%14c%15cccc%16c%17cccc%18c%19cc%20c%21cccc%22c%23cccc%24c%25ccc%26c%27ccc%28c(c1)c2c1c(c34)c2c5c3c6c4c(c87)c(c9%10)c5c%11c%12c6c7c%13c%14c8c(c%16%15)c(c%17%18)c9c%19c%10c%20c%11c(c%22%21)c(c%23%24)c%12c%25c%26c%13c%14c%27c%28c1c1c2c2c3c3c4c5c6c4c5c7c8c9c6c%10c7c%11c%12c%13c8c(c%141)c2c(c34)c(c78)c65]

The compound of formula C₁₃₂H₃₄ was prepared as described in Angew. Chem. Int. Ed. (1997) 36, 631.
C₂₂₂H₄₂ (graphene molecule of formula VII)

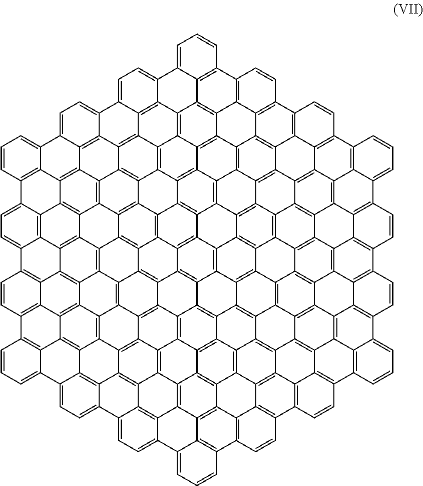

The compound of formula C₂₂₂H₄₂ was prepared as described in Angew. Chem. Int. Ed. (1997) 36, 1604.

Each of these graphene molecules was reacted with a halogen-donor compound in the presence of a Lewis acid. ICl was used as halogen-donor, and the Lewis acid was AlCl₃.

The graphene molecule C₄₂H₁₈ (I) was halogenated as follows:

A 50 ml flask was charged with 0.1 mmol (52 mg) of C₄₂H₁₈, 0.2 mmol (26 mg) of AlCl₃, 30 mmol (5 g) ICl and 35 ml of CCl₄, and then the reactants were stirred and refluxed at 80° C. for 48 h. After reaction, the products were poured into 30 ml ethanol to quench the reaction and precipitate the products. Then the suspension was filtered and precipitate was washed by ethanol, hydrochloric acid (1.0 mol/L), ion-free water and acetone, sequentially. After dried in vacuum, about 107 mg (0.097 mmol) yellow powder was obtained. The yield is about 97%.

The graphene molecule C₄₈H₁₈ (II) was halogenated as follows:

A 50 ml flask was charged with 0.1 mmol (60 mg) of C₄₈H₁₈, 0.2 mmol (26 mg) of AlCl₃, 30 mmol (5 g) ICl and 35 ml of CCl₄, and then the reactants were stirred and refluxed at 80° C. for 48 h. After reaction, the products were poured into 30 ml ethanol to quench the reaction and precipitate the products. Then the suspension was filtered and precipitate was washed by ethanol, hydrochloric acid (1.0 mol/L), ion-free water and acetone, sequentially. After dried in vacuum, about 103 mg (0.086 mmol) orange powder was obtained. The yield is about 85%.

The graphene molecule C₆₀H₂₂ (III) was halogenated as follows:

A 50 ml flask was charged with 0.1 mmol (75 mg) of C₆₀H₂₂, 0.25 mmol (34 mg) of AlCl₃, 30 mmol (5 g) ICl and 35 ml of CCl₄, and then the reactants were stirred and refluxed at 80° C. for 48 h. After that, the excess ICl and solvent CCl₄ were removed by rotary evaporator at 45° C. Dark red powder was obtained and washed with ethanol for 2 times. Then the product was purified by column chromoatography using chloroform/hexane (1:1) as eluent. The product was collected as the first component at solvent front. After evaporating the solvent and dried in vacuum, 143 mg dark red powder was obtained. The yield is about 95%.

The graphene molecule C₆₀H₂₄ (IV) was halogenated as follows:

A 50 ml flask was charged with 0.1 mmol (75 mg) of C₆₀H₂₄, 0.25 mmol (34 mg) of AlCl₃, 30 mmol (5 g) ICl and 35 ml of CCl₄, and then the reactants were stirred and refluxed at 80° C. for 48 h. After that, the excess ICl and solvent CCl₄ were removed by rotary evaporator at 45° C. Red powder was obtained and washed with ethanol for 2 times. Then the product was purified by column chromatography using chloroform as eluent. The product was collected as the first component at solvent front. After evaporating the solvent and dried in vacuum, 145 mg red powder was obtained. The yield is about 93%.

The graphene molecule C₉₆H₃₀ (V) was halogenated as follows:

A 50 ml flask was charged with 0.05 mmol (60 mg) of C₉₆H₃₀, 0.20 mmol (28 mg) of AlCl₃, 30 mmol (5 g) ICl and 35 ml of CCl₄, and then the reactants were stirred and refluxed at 80° C. for 48 h. After that, the excess ICl and solvent CCl₄ were removed by rotary evaporator at 45° C. Black powder was obtained and washed with ethanol for 2 times. Then the product was purified by column chromatography using chloroform as eluent. The product was collected as the first component at solvent front. After evaporating the solvent and dried in vacuum, 100 mg black powder was obtained. The yield is about 95%.

The graphene molecule C₁₃₂H₃₄ (VI) was halogenated as follows:

A 50 ml flask was charged with 0.015 mmol (25 mg) of C₁₃₂H₃₄, 0.20 mmol (28 mg) of AlCl₃, 30 mmol (5 g) ICl and 35 ml of CCl₄, and then the reactants were stirred and refluxed at 80° C. for 5 days. After reaction, the excess ICl and solvent CCl₄ were removed by rotary evaporator at 50° C. Black powder was obtained and washed with ethanol 2 times. Then the product was purified by column chromatography using chloroform/carbon disulfide (1:1) as eluent. The product was collected as the first component at solvent front. After evaporating the solvent and dried in vacuum, 34 mg black powder was obtained. The yield is about 83%.

The graphene molecule C₂₂₂H₄₂ (VII) was halogenated as follows:

A 50 ml flask was charged with 0.01 mmol (27 mg) of C₂₂₂H₄₂, 0.20 mmol (26 mg) of AlCl₃, 15 mmol (2.5 g) ICl and 35 ml of CCl₄, and then the reactants were stirred and refluxed at 80° C. for 60 h. After that, the excess ICl and solvent CCl₄ were removed by rotary evaporator at 45° C. Black powder was obtained and washed with ethanol 2 times. Then the product was purified by column chromatography using chloroform/carbon disulfide (1:1) as eluent. The product was collected as the first component at solvent front. After evaporating the solvent and dried in vacuum, 38 mg black powder was obtained. The yield is about 90%.

Mass spectra of the halogenated graphene molecules were recorded. The mass spectra were acquired by Bruker time of flight mass spectra coupled with matrix-assisted laser desorption ionic source (MALDI-TOF). The mass spectra of all halogenated graphene molecules show one major molecular mass peak, indicating the purity and defined structure of obtained chlorinated graphene molecules. The isotopic distribution pattern of molecular mass peaks of the chlorinated graphene molecules is in agreement with that calculated for molecular formulas (XIII) to (XIX) shown further below.

IR spectra were also measured on the halogenated graphene molecules. The IR spectra were acquired on a KBr crystal disc coated with the solid film of chlorinated graphene molecules. There is no C—H stretch signal in the IR spectra of those chlorinated graphene molecules prepared from compounds of formulas (I)-(IV) and (VII), validating their complete chlorine functionalization at the edge of the graphene molecules. Due to the high steric hindrance at double-fused bay edge configuration of compounds (V) and (VI), three and two hydrogen atoms remained respectively, which are clearly shown in the IR spectra.

The XPS spectra were measured on an ESCALAB 250 (Thermo-VG Scientific) equipped with an Al Ka monochromatic source using powder sample.

Mass spectra, IR spectra and XPS spectra confirm that halogenation was selectively effected at the edge of the graphene molecules while any halogenation on the aromatic basal plane was completely suppressed and the degree of halogenation at the edge was very high or even quantitative. Only those edge-bonded H-atoms which were sterically protected by a double-fused bay edge configuration were not substituted by Cl atoms.

From halogenation of the graphene molecule (I), the following edge-halogenated graphene molecule (XIII) was obtained:

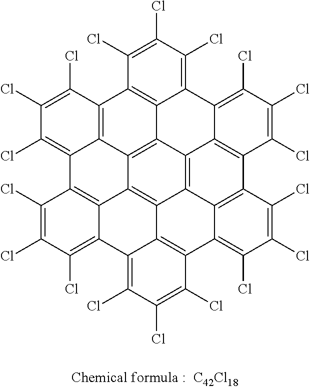 [SMILES: Clc1c(Cl)c2c3c(Cl)c(Cl)c(Cl)c4c5c(Cl)c(Cl)c(Cl)c6c7c(Cl)c(Cl)c(Cl)c8c9c(Cl)c(Cl)c(Cl)c%10c%11c(Cl)c(Cl)c(Cl)c%12c(c1Cl)c2c1c(c34)c(c56)c(c78)c(c%109)c1c%12%11]

From halogenation of the graphene molecule (II), the following edge-halogenated graphene molecule (XIV) was obtained:

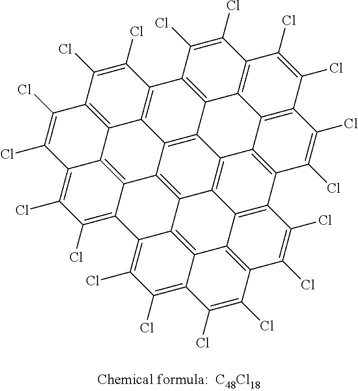 [SMILES: Clc1c(Cl)c2c(Cl)c(Cl)c3c4c(Cl)c(Cl)c5c(Cl)c(Cl)c6c(Cl)c(Cl)c7c8c(Cl)c(Cl)c9c(Cl)c(Cl)c%10c(Cl)c(Cl)c%11c%12c(Cl)c(Cl)c1c1c2c3c2c(c%121)c1c%11c%10c9c8c1c1c7c6c5c4c21]

From halogenation of the graphene molecule (III), the following edge-halogenated graphene molecule (XV) was obtained:

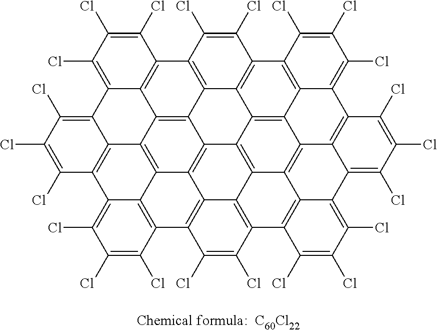 [SMILES: Clc1c(Cl)c2c3c(Cl)c(Cl)c(Cl)c4c5c(Cl)c(Cl)c6c7c(Cl)c(Cl)c(Cl)c8c9c(Cl)c(Cl)c(Cl)c%10c%11c(Cl)c(Cl)c(Cl)c%12c%13c(Cl)c(Cl)c%14c%15c(Cl)c(Cl)c(Cl)c%16c(c1Cl)c2c1c(c34)c2c5c6c3c(c87)c(c9%10)c(c%11%12)c4c%13c%14c(c1c%16%15)c2c34]

From halogenation of the graphene molecule (IV), the following edge-halogenated graphene molecule (XVI) was obtained:

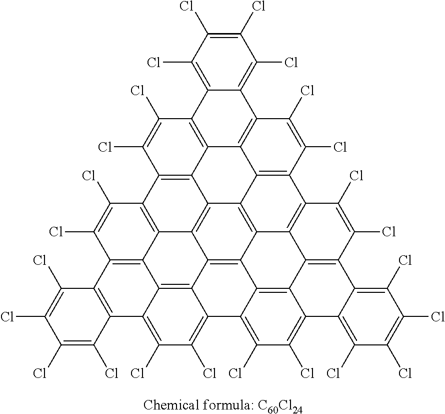 [SMILES: Clc1c(Cl)c(Cl)c2c(c1Cl)c1c(Cl)c(Cl)c3c4c(Cl)c(Cl)c5c6c(Cl)c(Cl)c(Cl)c(Cl)c6c6c(Cl)c(Cl)c7c8c(Cl)c(Cl)c9c%10c(Cl)c(Cl)c(Cl)c(Cl)c%10c%10c(Cl)c(Cl)c%11c%12c(Cl)c(Cl)c2c2c1c3c1c(c%122)c2c%11c%10c9c8c2c2c7c6c5c4c12]

From halogenation of the graphene molecule (V), the following edge-halogenated graphene molecule (XVII) was obtained:

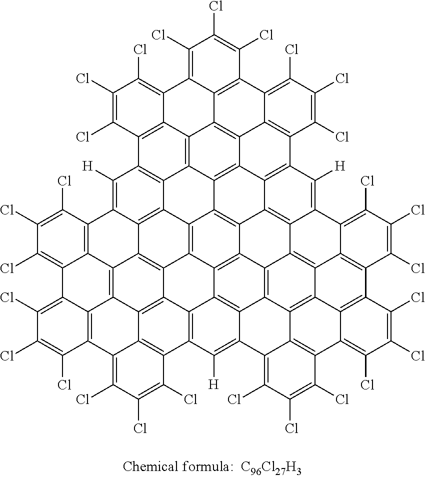 [SMILES: Clc1c(Cl)c2c3c(Cl)c(Cl)c(Cl)c4c5cc6c7c(Cl)c(Cl)c(Cl)c8c9c(Cl)c(Cl)c(Cl)c%10c%11c(Cl)c(Cl)c(Cl)c%12c%13cc%14c%15c(Cl)c(Cl)c(Cl)c%16c%17c(Cl)c(Cl)c(Cl)c%18c%19c(Cl)c(Cl)c(Cl)c%20c%21cc%22c%23c(Cl)c(Cl)c(Cl)c%24c(c1Cl)c2c1c(c43)c2c5c3c6c4c(c78)c(c9%10)c(c%12%11)c5c%13c6c%14c7c(c%15%16)c(c%18%17)c(c%20%19)c8c%21c9c%22c(c1c%23%24)c2c1c3c(c45)c6c(c87)c91]

From halogenation of the graphene molecule (VI), the following edge-halogenated graphene molecule (XVIII) was obtained:

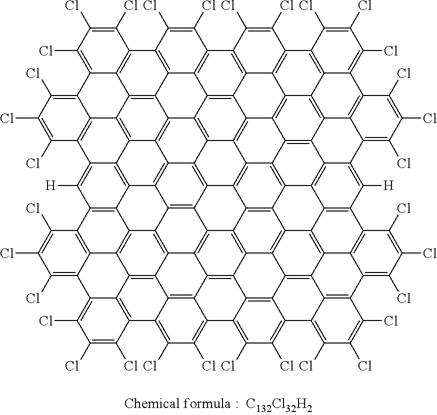 [SMILES: Clc1c(Cl)c2c3cc4c5c(Cl)c(Cl)c(Cl)c6c7c(Cl)c(Cl)c(Cl)c8c9c(Cl)c(Cl)c%10c%11c(Cl)c(Cl)c%12c%13c(Cl)c(Cl)c(Cl)c%14c%15c(Cl)c(Cl)c(Cl)c%16c%17cc%18c%19c(Cl)c(Cl)c(Cl)c%20c%21c(Cl)c(Cl)c(Cl)c%22c%23c(Cl)c(Cl)c%24c%25c(Cl)c(Cl)c%26c%27c(Cl)c(Cl)c(Cl)c%28c(c1Cl)c2c1c(c%28%27)c2c%26c%25c%25c%26c%24c%23c%23c(c%21%22)c(c%19%20)c%19c%18c%18c%17c%17c(c%16%15)c(c%14%13)c%13c%12c%11c%11c%12c%10c9c9c(c78)c(c56)c5c4c4c3c1c1c2c%25c2c3c%26c%23c%19c6c%18c7c%17c%13c%11c8c%10c%12c9c5c5c4c1c2c(c5%10)c(c63)c78]

From halogenation of the graphene molecule (VII), the following edge-halogenated graphene molecule (XIX) was obtained:

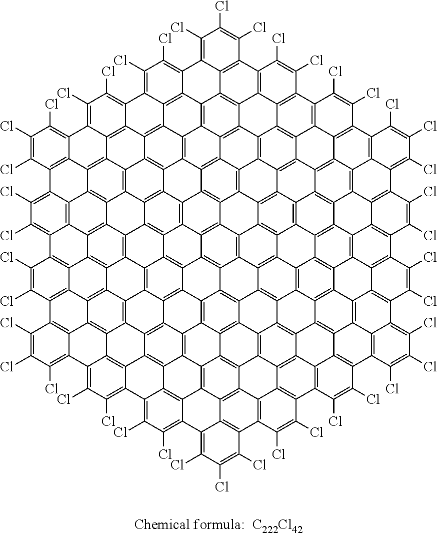

In further experiments, each of the halogenated graphene molecules (XIII) to (XVII) was crystallized from solution by solvent evaporation. On these crystalline forms of graphene molecules (XIII) to (XVIII), X-ray diffraction measurements were made. These XRD measurements confirmed the structures shown above.

Single crystals of (XIII) were grown from its carbon disulfide solution by solvent evaporation. The X-ray diffraction was measured on a STOE diffractometer using a graphite-monochromated Cu Kα radiation source (1.54178 Å).

Crystal Data:

C₄₂Cl₁₈.(CS2)₂, M=1294.78, triclinic, a=9.1469(18) Å, b=10.368(2) Å, c=12.092(2) Å, α=86.48(3)°, β=88.75(3)°, γ=75.41(3)°, V=1107.6(4) Å3, T=193(2)

K, space group P-1, Z=4, μ(Cu Kα)=12.292, 13412 reflections measured, 3603 unique (Rint=0.1935) which were used in all calculations. The final wR2 was 0.3097 (all data) and R1 was 0.0959 (>2sigma(I)).

Single crystals of (XIV) were grown form carbon disulfide solution by solvent evaporation. The X-ray diffraction was measured on an Oxford Supernova diffractometer using a graphite-monochromated Cu Kα radiation source (1.54178 Å).

Crystal Data: C₄₈Cl₁₈, M=1214.58, monoclinic, a=12.861(2) Å, b=28.435(4) Å, c=10.629(3) Å, β=97.155(18)°, V=3856.7(13) Å3, T=173(2) K, space group C2/c (no. 15), Z=4, μ(CuKα)=12.097, 9190 reflections measured, 3161 unique (Rint=0.0430) which were used in all calculations. The final wR2 was 0.2542 (all data) and R1 was 0.0825 (>2sigma(I)).

Single crystals of (XV) were grown from its carbon disulfide/chloroform (1:1) solution by solvent evaporation. Then crystal was measured on Bruker diffractometer using a graphite-monochromated Mo Kα radiation source (0.71073 Å).

Crystal Data: C₆₀Cl₂₂, M=1500.50, monoclinic, a=27.683(6) Å, b=21.998(4) Å, c=21.006(4) Å, β=91.15(3)°, V=12790(4) Å³, T=173(2) K, space group C2/c (no. 15), Z=8, μ(MoKα)=0.976, 35168 reflections measured, 12556 unique (Rint=0.0756) which were used in all calculations. The final wR2 was 0.1397 (all data) and R1 was 0.0651 (>2sigma(I)).

Single crystals of (XVI) were grown from its toluene solution by solvent evaporation. The X-ray diffraction was measured on an Oxford Supernova diffractometer using a graphite-monochromated Cu Kα radiation source (1.54178 Å).

Crystal Data: C₆₀Cl₂₄, M=1571.40, monoclinic, a=20.4128(7) Å, b=22.9777(6) Å, c=15.0794(5) Å, β=108.949(4)°, V=6689.5(4) Å³, T=173(2) K, space group C2/c (no. 15), Z=4, μ(CuKα)=9.277, 12430 reflections measured, 5897 unique (Rint=0.0301) which were used in all calculations. The final wR2 was 0.0959 (all data) and R1 was 0.0369 (>2sigma(I)).

Single crystals of (XVII) were grown from its chloroform/cyclohexane solution by solvent evaporation. The X-ray diffraction was measured on an Oxford Supernova diffractometer using a graphite-monochromated Cu Kα radiation source (1.54178 Å).

Crystal Data: C₉₆H₃Cl₂₇, M=2113.13, monoclinic, a=36.715(4) Å, b=22.1591(12) Å, c=24.607(3) Å, β=116.242(14), V=17956(3) Å3, T=173(2), space group C2/c (no. 15), Z=8, μ(CuKα)=7.891, 32778 reflections measured, 14891 unique (Rint=0.0532) which were used in all calculations. The final wR2 was 0.1681 (all data) and R1 was 0.0620 (>2sigma(I)).

### II. Preparation of Halogenated Structurally Defined Graphene Nanoribbons

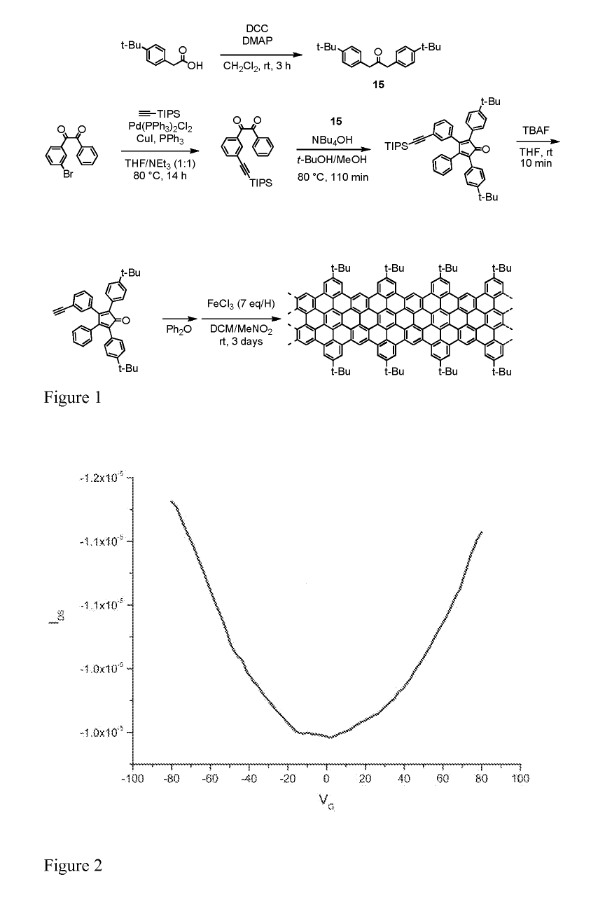

A structurally defined graphene nanoribbon was prepared according to the scheme shown in FIG. 1 and then used as the starting graphene material to be halogenated.

The starting graphene nanoribbon had a molecular weight of around 23,000 Da and a well-defined structure (i.e. characterized by a repeating unit RU so that the structure of the GNR can be represented as [RU]ₙ) which can be illustrated by the following formula:

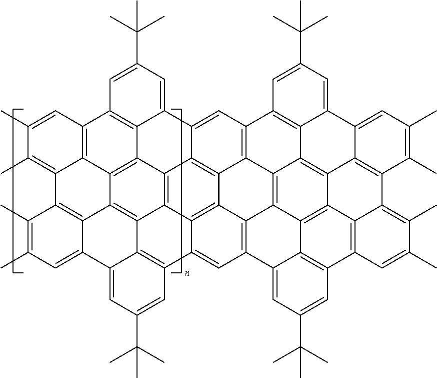

The structurally defined graphene nanoribbon DGNR (Defined Graphene Nano Ribbon) was halogenated according to the following procedure:

A 100 ml flask was charged with 25 mg of GNR, 0.2 mmol (26 mg) of AlCl₃, 30 mmol (5 g) ICl and 70 ml of CCl₄, and then the reactants were stirred and refluxed at 80° C. for 4 days. After reaction, 30 ml ethanol was added to quench the reaction. The solvent was removed by rotary evaporator at 50° C. Then 30 ml ethanol was added. After sonicated for 5 min, the suspension was filtered. The precipitate was washed with ethanol, hydrochloric acid (1.0 mol/L), ion-free water and acetone, sequentially. After dried in vacuum, about 33 mg (0.086 mmol) dark violet powder was obtained. The yield is about 85%.

The XPS spectra were measured on an ESCALAB 250 (Thermo-VG Scientific) equipped with an Al Ka monochromatic source using powder sample.

IR spectra and XPS analysis made on the halogenated DGNR confirmed that halogenation was selectively effected at the edge of the DGNR, and the edge-bonded tert-butyl groups as well as the hydrogen atoms which are in ortho-position to the tert-butyl group were substituted by halogen atoms, whereas the hydrogen atoms which are sterically protected by the “double-fused bay edge configuration” remain unsubstituted. Just like the starting graphene nanoribbon, the halogenated graphene nanoribbon has a very well-defined structure characterized by an edge-halogenated repeating unit.

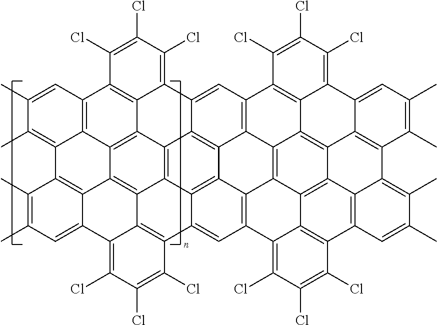

### III. Preparation of a Halogenated Graphene Nanoribbon that does not have a Repeating Unit and of a Halogenated Graphene

### III.1 the Edge-Halogenated Graphene

The starting graphene was prepared by reducing graphene oxide with hydrazine.

25 mg of the graphene, 0.2 mmol (26 mg) of AlCl₃, 30 mmol (5 g) ICl and 35 ml of CCl₄ were added into a 50 ml flask. The reactants were stirred and refluxed at 80° C. for 4 days. After reaction, 30 ml ethanol was added to quench the reaction. After sonicated for 5 min, the suspension was filtered. The precipitate was washed by ethanol, hydrochloric acid (1.0 mol/L) and ion-free water, sequentially.

Scanning electron microscopy confirmed that the morphology of the flakes was maintained after chlorination and XPS analysis showed that halogenation was selectively effected at the edges of the graphene while any halogenation on the aromatic basal plane was suppressed.

III.2 the Halogenated Graphene Nanoribbon that does not have a Repeating Unit

The starting graphene nanoribbon GNR was prepared by unzipping multi-wall carbon nanotubes. With this top-down approach, a starting GNR is obtained which does not have a repeating unit.

15 mg of GNR, 0.2 mmol (26 mg) of AlCl₃, 30 mmol (5 g) ICl and 35 ml of CCl₄ was added into a 50 ml flask. The reactants were stirred and refluxed at 80° C. for 4 days. After reaction, 30 ml ethanol was added to quench the reaction. After sonicated for 5 min, the suspension was filtered. The precipitate was washed by ethanol, hydrochloric acid (1.0 mol/L) and ion-free water, sequentially.

Scanning electron microscopy confirmed that the morphology of the ribbons was maintained after chlorination and XPS analysis showed that halogenation was selectively effected at the edges of the graphene while any halogenation on the aromatic basal plane was suppressed.

### IV. Fabrication of a Field Effect Transistor Device Using Chlorinated Graphene

A single sheet FET device was fabricated from the chlorinated graphene prepared in III.1 and compared with a device based on the non-chlorinated graphene. Both devices show the similar hole mobility of around 10 cm²V⁻¹s⁻¹, whereas the electron mobility of the chlorinated graphene increases from 1.0 cm²V⁻¹s⁻¹ (for non-chlorinated graphene) to 5.5 cm²V⁻¹s⁻¹.

FIG. 2 shows the I_{SD}-V_{G} characteristic curve of single layer FET devices of the edge-chlorinated graphene.

1. A process for edge-halogenation of a graphene material, wherein the graphene material, which is selected from a graphene, a graphene nanoribbon, a graphene molecule, or a mixture thereof, is reacted with a halogen-donor compound in the presence of a Lewis acid, so as to obtain an edge-halogenated graphene material.

2. The process according to claim 1, wherein the graphene material has edge-bonded residues R_{E} which are selected from hydrogen, a substituted or unsubstituted alkyl group, a substituted or unsubstituted aryl group, or any combination thereof.

3. The process according to claim 1, wherein the graphene molecule is a polycyclic aromatic compound having from 8 to 200 fused aromatic rings.

4. The process according to claim 3, wherein the graphene molecule is selected from one or more of the following compounds (I) to (VII): 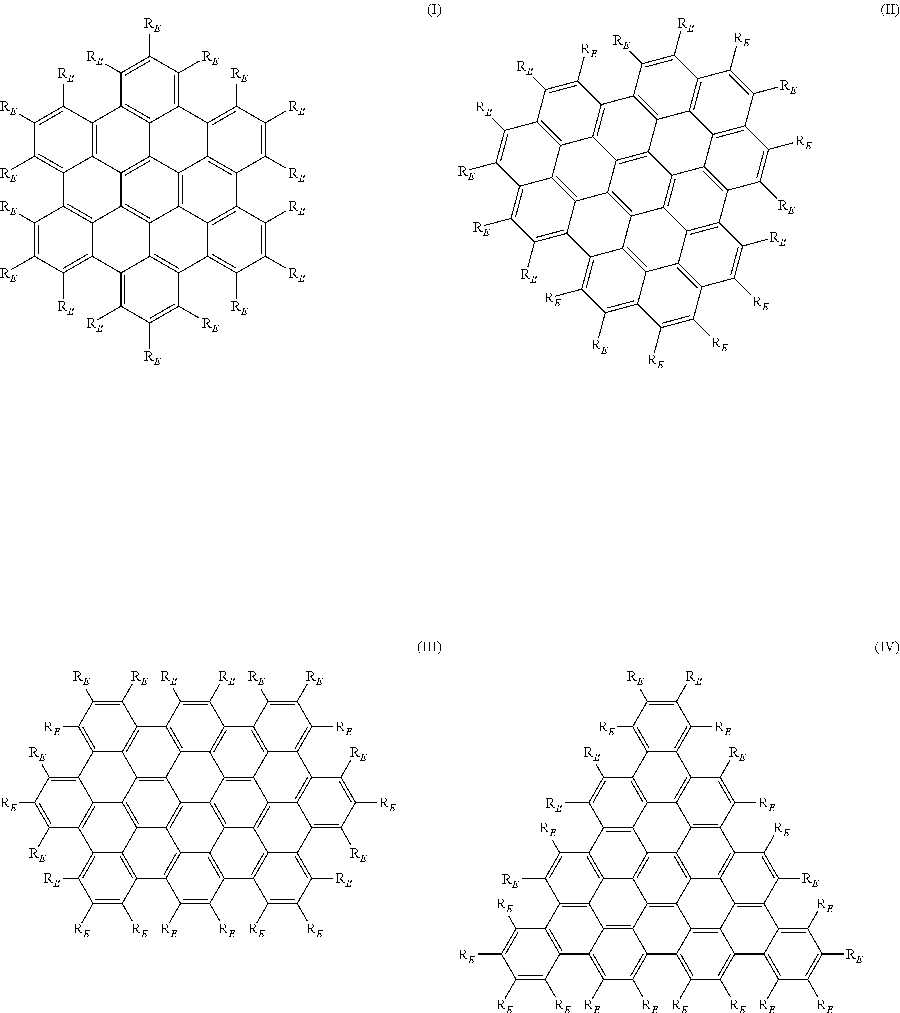 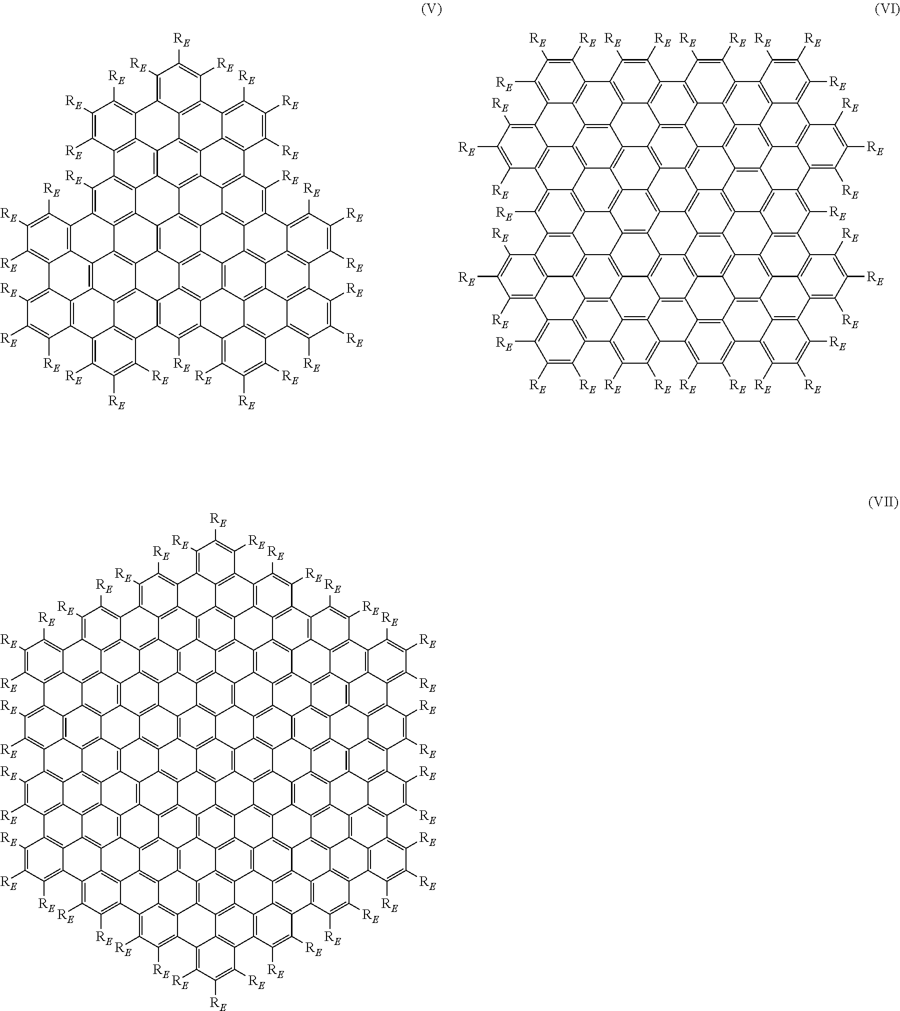
wherein the edge-bonded residues R_{E} are selected from hydrogen, a substituted or unsubstituted alkyl group, a substituted or unsubstituted aryl group, or any combination thereof.

5. The process according to claim 1, wherein the graphene nanoribbon has a maximum width which is less than 50 nm.

6. The process according to claim 1, wherein the halogen-donor compound is selected from an interhalogen compound, S₂Cl₂, SOCl₂, a mixture of S₂Cl₂ and SOCl₂, SO₂Cl₂, Cl₂, Br₂, F₂, I₂, PCl₃, PCl₅, POCl₃, POCl₅, POBr₃, N-bromo succinimide, N-chloro succinimide, or any mixture thereof.

7. The process according to claim 6, wherein the interhalogen compound is a compound having the following formula (VIII):
XYₙ (VIII)
wherein
n is 1, 3, 5, or 7;
X and Y, which are different, are selected from F, Cl, Br and I.

8. The process according to claim 1, wherein Lewis acid is selected from a compound of formulas (IX) to (XII):
AX₃ (IX)
wherein A is Al, Fe, Sm, Sc, Hf, In, Y or B, and X is halogen (preferably F, Cl, Br, P) or a trifluorosulfonate;
AX₅ (X)
wherein A is P, Sb, Mo, or As, and X is halogen;
AX₄ (XI)
wherein A is Ti or Sn, and X is halogen;
AX₂ (XII)
wherein A is Mg, Zn, Cu or Be, and X is halogen or a trifluorosulfonate.

9. The process according to claim 1, wherein at least one of a) the molar ratio of the edge-bonded residues R_{E} of the graphene, the graphene nanoribbon or the graphene molecule to the Lewis acid is within the range of from 100/1 to 1/5; and b) the molar ratio of the edge-bonded residues R_{E} of the graphene, the graphene nanoribbon or the graphene molecule to the halogen-donor compound is within the range of from 1/1 to 1/100.

10. The process according to claim 1, wherein the process is carried out in an organic liquid.

11. A halogenated graphene material, which is obtained by the process according to claim 1.

12. A halogenated graphene material comprising an aromatic basal plane and an edge, wherein at least 65 mole % of the residues R_{E} covalently attached to the edge of the halogenated graphene material are halogen atoms HA_{E}, and the edge-bonded halogen atoms HA_{E} represent at least 95 mole % of all halogen atoms being present in the halogenated graphene material, and wherein the halogenated graphene material is selected from a halogenated graphene, a halogenated graphene nanoribbon and a halogenated graphene molecule.

13. The halogenated graphene material according to claim 11, wherein the halogenated graphene molecule has one of the following formulas (XIII), (XIV), (XV), (XVI), (XVII), (XVIII), and (XIX):

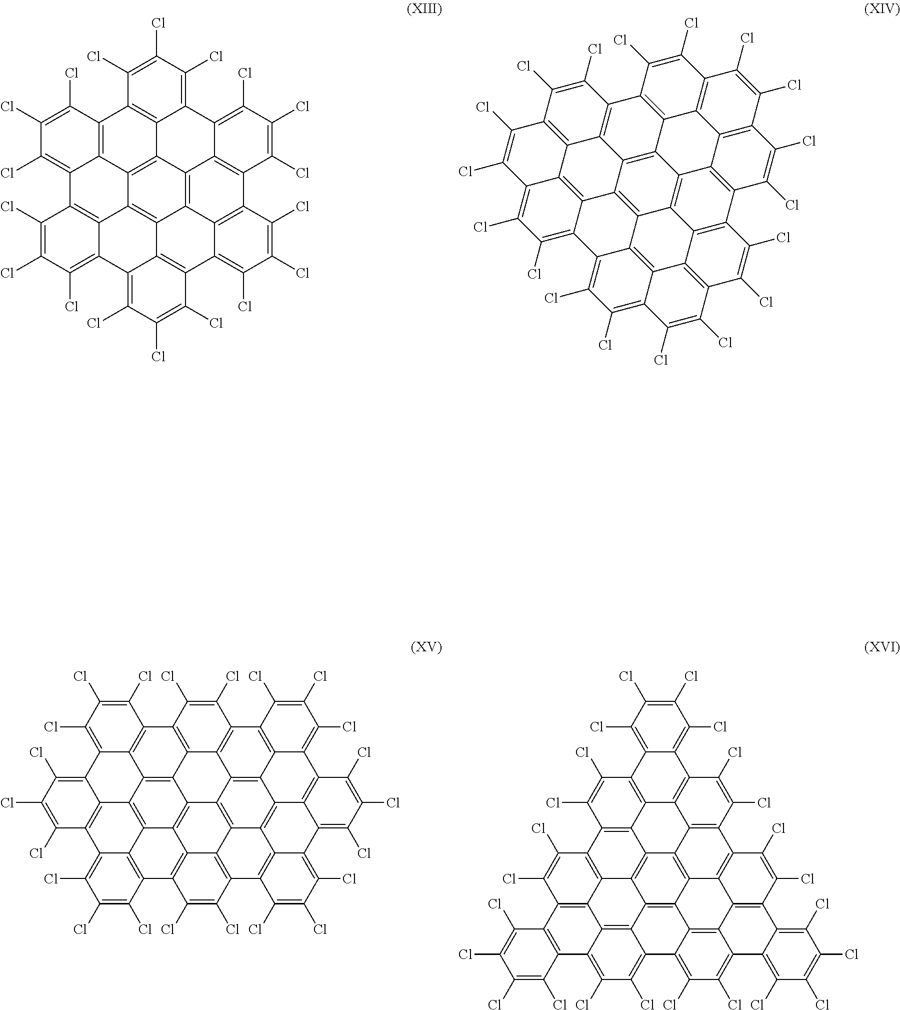

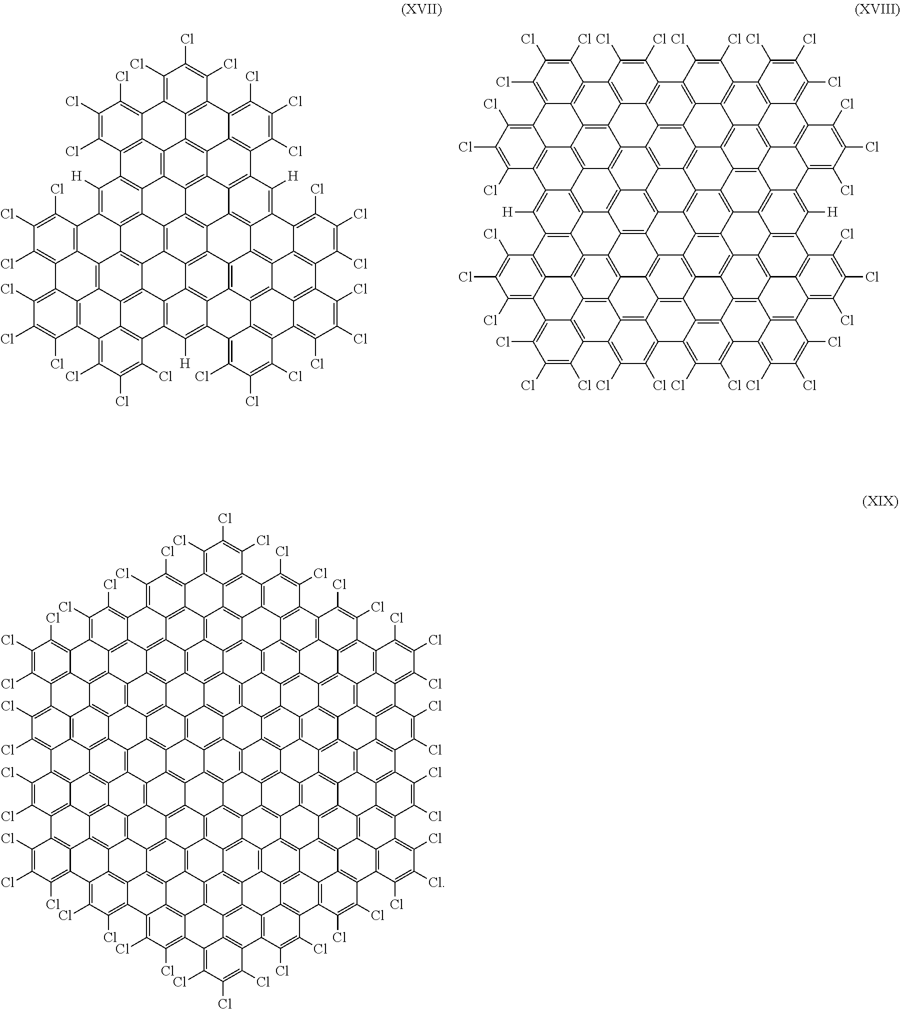

14. The halogenated graphene material according to claim 11, wherein at least one of a) the halogenated graphene nanoribbon has a maximum width of less than 50 nm, and b) at least a segment of the halogenated graphene nanoribbon is made of [RU]ₙ, wherein RU is a repeating unit and 2≦n≦2500.

15. A composition comprising the graphene material according to claim 11 dissolved or dispersed in a liquid medium.

16. An electronic, optical, or optoelectronic device comprising a semiconductor film which comprises the graphene material according to claim 11.

17. The device according to claim 16, wherein the device is an organic field effect transistor device, an organic photovoltaic device, or an organic light-emitting diode.

18. Use of the graphene material according to claim 11 in an electronic, optical, or optoelectronic device.
